(19) United States
(12) Patent Application Publication
(10) Pub. No.: US 2012/0140179 A1
(43) Pub. Date: Jun. 7, 2012

(54) TOMOGRAPHY APPARATUS, CONTROL METHOD FOR THE SAME, PROGRAM, AND STORAGE MEDIUM

(75) Inventors: Kazuhiro Miyasa, Yokohama-shi (JP); Akihiro Katayama, Zama-shi (JP)

(73) Assignee: CANON KABUSHIKI KAISHA, Tokyo (JP)

(21) Appl. No.: 13/389,588

(22) PCT Filed: Jul. 23, 2010
(86) PCT No.: PCT/JP2010/062968

§ 371 Date: Feb. 8, 2012

(30) Foreign Application Priority Data
Aug. 11, 2009 (JP) 2009-186779

(51) Int. Cl. A61B 3/14

(52) U.S. Cl. 351/206; 351/246

(57) ABSTRACT

In a tomography apparatus that captures tomograms of a fundus through optical coherence tomography, when a measurement area in which tomograms are to be captured is set on the fundus, tomograms are acquired with use of the optical coherence tomography at a plurality of predetermined positions in the set measurement area, the number of predetermined positions being smaller than in the case of imaging for diagnosis. The acquired tomograms are then displayed inline on the screen of a display apparatus in real-time.

### TECHNICAL FIELD

The present invention relates to a tomography apparatus and a control method for the same, and in particular relates to a tomography apparatus used in ophthalmic practice etc. and a control method for the same.

### BACKGROUND ART

Eye tomography apparatus that employ OCT (Optical Coherence Tomography) and the like have gained attention in recent years due to enabling three-dimensional observation of the internal condition of retina layers, and being useful for more accurately diagnosing illnesses.

FIGS. 5A and 5B are illustrative diagrams respectively showing the OCT measurement area on a fundus and corresponding retina tomograms. In FIG. 5A, 501 denotes a fundus image, and R_{XY} denotes a two-dimensional OCT measurement area on the fundus plane (the x axis being the horizontal direction, and the y axis being the vertical direction). In the example in FIG. 5A, R_{XY} is a rectangular area. Also, T₁ to Tₙ shown in FIG. 5B are two-dimensional tomograms (B-Scan images) of the macular portion that were obtained by performing imaging in the measurement area R_{XY} in the depth direction of the retina. Each of the tomograms is made up of multiple scan lines (hereinafter, referred to as “A-scan lines”) scanning in the depth direction of the retina. The z axis represents this A-scan direction, and R_{Z} denotes the one-dimensional OCT measurement area in the depth direction in the z axis direction. With imaging that employs OCT, the measurement area R_{XY} set on the fundus is successively raster scanned (scanning in the x axis direction being referred to as “main scanning”, and scanning in the y axis direction being referred to as “sub scanning”), thus acquiring three-dimensional data for the group of tomograms at one time. Also, M denotes the fovea, A denotes the inner limiting membrane, and B denotes the retinal pigment epithelium boundary. The area of retina layers between the inner limiting membrane A and the retinal pigment epithelium boundary B is extremely useful when making a diagnosis with use of OCT tomograms since the anatomical characteristics of illnesses such as glaucoma and age-related macular degeneration, which are the main causes of vision loss, appear in this area. For this reason, when capturing a tomogram, it is critical to perform imaging such that this area is not cut off at the upper edge or lower edge in the depth direction of the tomogram.

When observing a test-subject eye before capturing three-dimensional data with a common OCT apparatus, one or more tomograms that pass through only the center of the measurement area R_{XY} are acquired in real-time and displayed. Doing this enables visually checking whether the entire area of retina layers fits in the tomogram, and appropriately adjusting the imaging position. Also, Japanese Patent Laid-Open No. 2008-154939 discloses a technique in which a single tomogram acquired while observing a test-subject eye is analyzed, and a determination is made as to whether the retina layers appear in the tomogram, and thus the imaging position is automatically adjusted so that the retina layers appear in the tomogram.

However, with the aforementioned technique, the photographer or computer observes only several tomograms that pass through the center of the measurement area R_{XY}, and therefore it has not been possible to determine during observation of the test-subject eye whether all of the retina layers will appropriately fit in the three-dimensional data that will be captured thereafter. In particular, in the case of imaging a nearsighted eye whose retina layers are steeply curved, even if all of the retina layers fit in the tomogram that passes through the center of the measurement area R_{XY} during observation of the test-subject eye, there has been the possibility that all of the retina layers will not appropriately fit in a tomogram at a position distanced from the center. In such a case, the imaging will fail, thus resulting in the need to re-capture the tomogram.

### SUMMARY OF INVENTION

The present invention has been achieved in light of the aforementioned problems, and according to a typical embodiment of the present invention, it is possible in an imaging apparatus that employs optical coherence tomography to easily and appropriately set an imaging position in the depth direction of a tomogram in a measurement area that has been set.

According to one aspect of the present invention, there is provided a control method for a tomography apparatus that captures a tomogram of a fundus through optical coherence tomography, the control method comprising: a setting step of setting, on the fundus, a measurement area in which a tomogram is to be captured; an acquisition step of acquiring tomograms with use of the optical coherence tomography at a plurality of predetermined positions in the measurement area, the number of the predetermined positions being smaller than that in a case of imaging for diagnosis; and a display control step of displaying the tomograms acquired in the acquisition step inline on a screen of a display apparatus in real-time.

According to another aspect of the present invention, there is provided a control method for a tomography apparatus that captures a tomogram of a fundus through optical coherence tomography, the control method comprising: a setting step of setting, on the fundus, a measurement area in which a tomogram is to be captured; an acquisition step of acquiring tomograms with use of the optical coherence tomography at a plurality of predetermined positions in the measurement area, the number of the predetermined positions being smaller than that in a case of imaging for diagnosis; an extraction step of extracting a retina layer from each of the tomograms acquired in the acquisition step; and an adjustment step of, based on positions in a depth direction in the tomograms of the retina layers extracted in the extraction step, adjusting a position of a reference mirror used in the optical coherence tomography so that the retina layers do not protrude outside an imaging area in the depth direction.

According to still another aspect of the present invention, there is provided a tomography apparatus that captures a tomogram of a fundus through optical coherence tomography, comprising: a setting unit that sets, on the fundus, a measurement area in which a tomogram is to be captured; an acquisition unit that acquires tomograms with use of the optical coherence tomography at a plurality of predetermined positions in the measurement area, the number of the predetermined positions being smaller than that in a case of imaging for diagnosis; and a display control unit that displays the tomograms acquired by the acquisition unit inline on a screen of a display apparatus in real-time.

According to yet another aspect of the present invention, there is provided a tomography apparatus that captures a tomogram of a fundus through optical coherence tomography, comprising: a setting unit that sets, on the fundus, a measurement area in which a tomogram is to be captured; an acquisition unit that acquires tomograms with use of the optical coherence tomography at a plurality of predetermined positions in the measurement area, the number of the predetermined positions being smaller than that in a case of imaging for diagnosis; an extraction unit that extracts a retina layer from each of the tomograms acquired by the acquisition unit; and an adjustment unit that, based on positions in a depth direction in the tomograms of the retina layers extracted by the extraction unit, adjusts a position of a reference mirror used in the optical coherence tomography so that the retina layers do not protrude outside an imaging area in the depth direction.

Further features of the present invention will become apparent from the following description of exemplary embodiments with reference to the attached drawings.

### BRIEF DESCRIPTION OF DRAWINGS

FIG. 1 is a diagram showing a functional configuration of a tomography apparatus according to an embodiment.

FIG. 2 is a diagram showing a device configuration and functional configuration of a tomogram acquisition unit 103.

FIG. 3 is a flowchart showing a processing procedure according to the embodiment.

FIG. 4 is a flowchart showing a processing procedure of step S310.

FIGS. 5A and 5B are diagrams respectively showing an OCT measurement area on a fundus and three-dimensional data that has been captured.

FIG. 6A is a diagram showing tomogram acquisition positions.

FIG. 6B is a diagram showing tomograms that have been acquired.

FIG. 7 is a diagram showing an example of a display method for displaying tomogram image data (without cutoff) inline.

FIG. 8 is a diagram showing an example of a display method for displaying tomogram image data (with cutoff) inline.

FIG. 9 is a diagram showing warning display that is performed if an inner limiting membrane has been cut off.

FIG. 10 is a diagram showing warning display that is performed in a case where a retinal pigment epithelium boundary has been cut off.

FIGS. 11A and 11B are diagrams showing the positional relationship between a tomogram and estimated lines of retina layers that have been cut off.

FIG. 12 is a diagram showing a device configuration of the tomography apparatus according to the embodiment.

FIGS. 13A to 13F are diagrams showing center positions and edge portion positions corresponding to measurement areas.

FIGS. 14A and 14B are diagrams for illustrating cutoff of retina layers.

### DESCRIPTION OF EMBODIMENTS

### Embodiment 1

In the present embodiment, when capturing a tomographic image of a test-subject eye through optical coherence tomography (hereinafter, referred to as “OCT”), tomograms are displayed while repeatedly scanning multiple sites in a measurement area, thereby enabling performing adjustment so that the entirety of the measurement target fits in images to be captured. More specifically, with a tomography apparatus according to the present embodiment, when observing the test-subject eye before capturing three-dimensional data for diagnosis through OCT, tomograms are acquired at the center position and edge portion positions of a measurement area R_{XY} of the test-subject eye, and the acquired tomograms are displayed inline on a confirmation screen in real-time. Here, since the three-dimensional shape of the retina can be approximated to an ellipsoidal body, there is the property that as the distance outward from the center of the measurement area R_{XY} increases, the difference between the center and a position in the depth (z axis) direction of the retina increases monotonically. According to this property, it is possible to observe the condition of tomograms at the center position and edge portion positions of the measurement area R_{XY}, which enables always allowing the photographer to be aware of whether all of the retina layers will fit in three-dimensional data that will be captured thereafter. The center position and edge portion positions of the measurement area R_{XY} referred to here are respectively the position that passes through the center of the measurement area R_{XY}, and positions including the area at the most outward edges of the measurement area R_{XY}. Several specific examples will be described below.

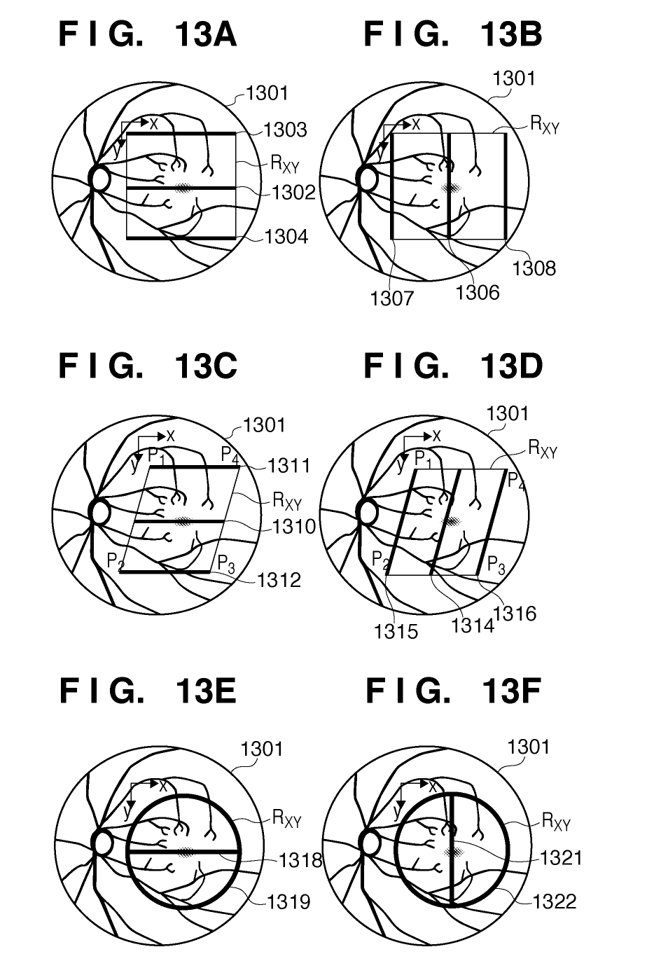

FIGS. 13A to 13F are diagrams showing center positions and edge portion positions corresponding to the measurement area R_{XY}. FIGS. 13A and 13B are two types of diagrams showing the center position and edge portion positions in the case where the measurement area R_{XY} is a rectangle. FIGS. 13C and 13D are two types of diagrams showing the center position and edge portion positions in the case where the measurement area R_{XY} is a parallelogram. FIGS. 13E and 13F are two types of diagrams showing the center position and an edge portion position in the case where the measurement area R_{XY} is a circle. In the diagrams 13A to 13F, 1301 denotes a fundus image, and R_{XY} denotes a two-dimensional measurement area. Also, 1302, 1306, 1310, 1314, 1318, and 1321 denote center positions of the measurement area R_{XY}. Additionally, 1303 and 1304, 1307 and 1308, 1311 and 1312, 1315 and 1316, and 1319 and 1322 denote edge portion positions. Furthermore, in FIGS. 13C and 13D, P₁, P₂, P₃, and P₄ denote the four vertices of the measurement area R_{XY}.

Here, the center positions and edge portion positions in these figures are all positions that meet the aforementioned definitions. In the case where the measurement area R_{XY} is a rectangle, the center position and edge portion positions may be line segments parallel with the x axis as shown in FIG. 13A, or may be line segments parallel with the y axis as shown in FIG. 13B. In the case where the measurement area R_{XY} is a parallelogram, the center position and edge portion positions may be line segments parallel with a side P₁P₄ as shown in FIG. 13C, or may be line segments parallel with a side P₁P₂ as shown in FIG. 13D.

In the case where the measurement area R_{XY} is a circle, the center position of the measurement area R_{XY} may be a line segment parallel with the x axis as shown in FIG. 13E, or may be a line segment parallel with the y axis as shown in FIG. 13F. Here, the edge portion position is a position corresponding to the circumference of the measurement area R_{XY}. In other words, a tomogram obtained by circular scanning is used. Although the present embodiment is described below taking the specific example of the case shown in FIG. 13A, the shape of the measurement area and the center position and edge portion positions are not limited to this example.

By displaying tomograms at the center position and two edge portion positions in real-time, a user can easily determine whether a retina layer protrudes outside the measurement area in the depth direction (has been cut off in relation to the depth direction). Also, since the OCT reference mirror can be moved while viewing tomograms at the center portion and two edge portions that are displayed in real-time, the user can easily set the reference mirror to an appropriate position. Also, whether a retina layer has been cut off in the depth direction of the measurement area in the tomogram is detected, and if a retina layer has been cut off, a warning to that effect is presented, thereby helping making the photographer aware that a retina layer has been cut off. Here, cutoff in the depth direction of a retina layer is detected by, for example, determining whether the retina layer touches or intersects the upper side or lower side of the tomogram. Furthermore, the ability to detect the position of the retina layer in the tomogram and automatically adjust the measuring depth in the z axis direction so that the retina layer is not cut off in the tomogram is provided, thereby mitigating the burden on the photographer, and furthermore preventing imaging mistakes. A specific example will be described below.

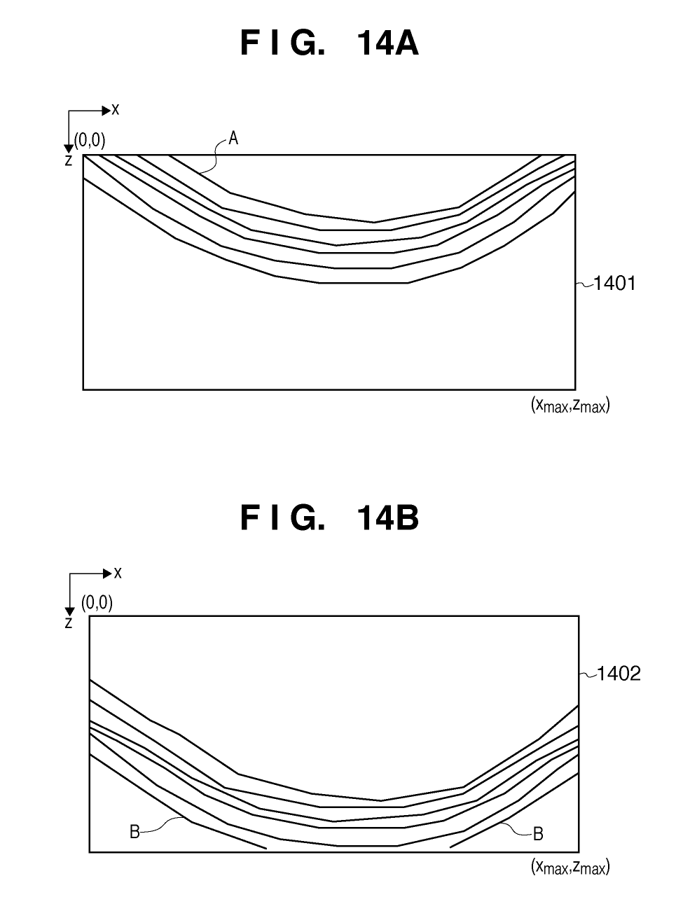

FIGS. 14A and 14B are diagrams for illustrating cutoff of retina layers. FIGS. 14A and 14B are diagrams respectively showing examples in which retina layers have been cut off at the upper side and the lower side of tomograms. Here, 1401 and 1402 that are respectively shown in FIGS. 14A and 14B denote tomograms in which retina layers have been cut off in the images shown in these cases. In these figures, the x axis is the main scanning direction, and the z axis is the A-scan direction. As shown in these figures, the range of coordinates in the tomograms is 0≦x≦xₘₐₓ and 0≦z≦zₘₐₓ. In FIG. 14A, A denotes the inner limiting membrane of the fundus. In FIG. 14A, the inner limiting membrane A intersects the straight line at z=0 (upper side of the tomogram), and therefore FIG. 14A shows the condition in which the inner limiting membrane A has been cut off. In FIG. 14B, B denotes the retinal pigment epithelium boundary. In FIG. 14B, the retinal pigment epithelium boundary B intersects the straight line at z=zₘₐₓ (lower side of the tomogram), and therefore FIG. 14B shows the condition in which the retinal pigment epithelium boundary B has been cut off. In this way, cutoff of a retina layer in the present embodiment specifically refers to cutoff of the inner limiting membrane or the retinal pigment epithelium boundary.

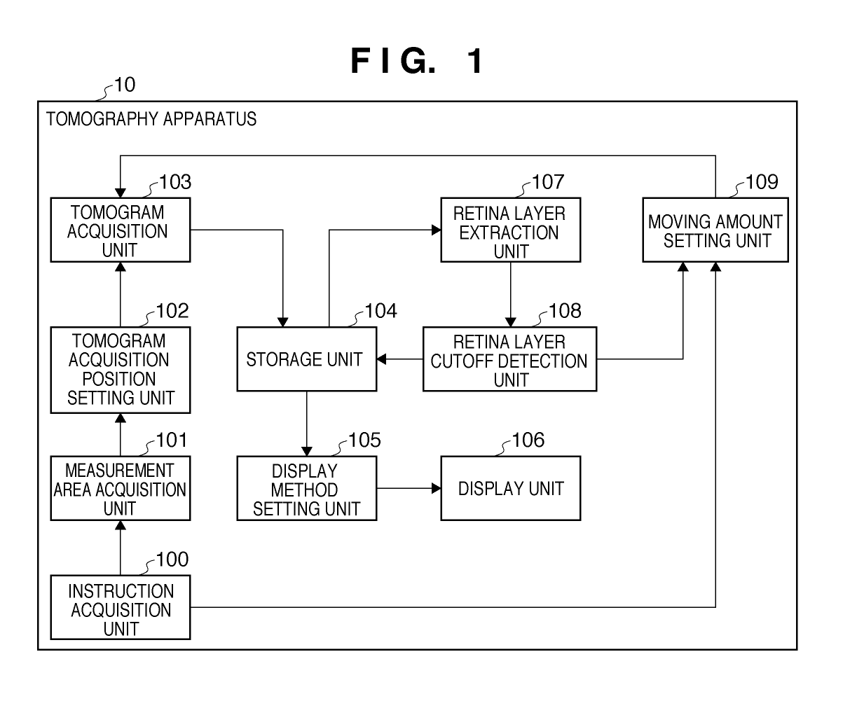

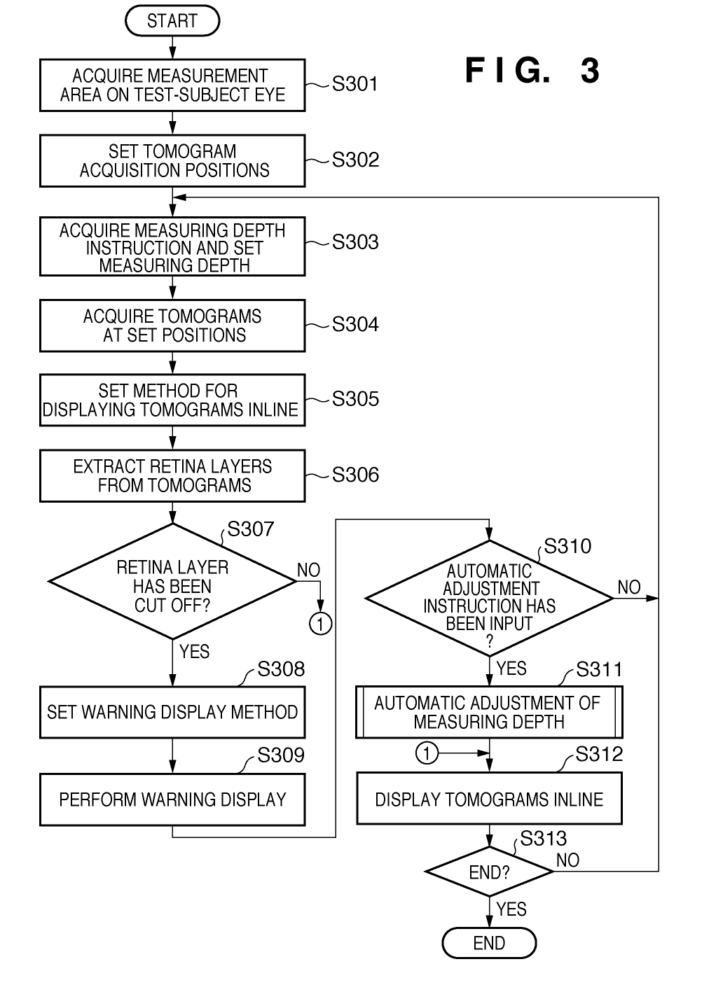

Next is a description of a configuration of the tomography apparatus 10 according to the present embodiment and specific processing procedures executed by the tomography apparatus 10, with reference to the block diagram in FIG. 1 and the flowchart in FIG. 3.

In step S301, a measurement area acquisition unit 101 acquires, from an instruction acquisition unit 100, instruction information from an operator that is for setting the two-dimensional measurement area R_{XY} on the fundus of a test subject, and specifies the measurement area R_{XY}. This instruction information is input by the operator via a keyboard or mouse (not shown) with which the tomography apparatus 10 is provided. As one example of the instruction regarding the measurement area R_{XY} on the fundus, the measurement area acquisition unit 101 acquires an instruction (defined as instruction 1) such as a designation of a site or position on the fundus targeted for tomogram acquisition. Then, the measurement area acquisition unit 101 specifies the measurement area R_{XY} that is a rectangle based on the content of this instruction 1. The specified measurement area R_{XY} is transmitted to a tomogram acquisition position setting unit 102.

In step S302, the tomogram acquisition position setting unit 102 acquires the measurement area R_{XY} from the measurement area acquisition unit 101, and sets positions where tomograms are to be acquired (hereinafter, referred to as tomogram acquisition positions P) within this measurement area. A predetermined number of positions are set as the tomogram acquisition positions P, and this number is smaller than that in the case of performing imaging for diagnosis. In this step, for example, the center position and edge portion positions of the measurement area R_{XY} are set as the tomogram acquisition positions P. The tomogram acquisition positions P are of course not limited to this combination. For example, five tomogram acquisitions positions P may be set by adding an acquisition position between the center and each of the two edge portions.

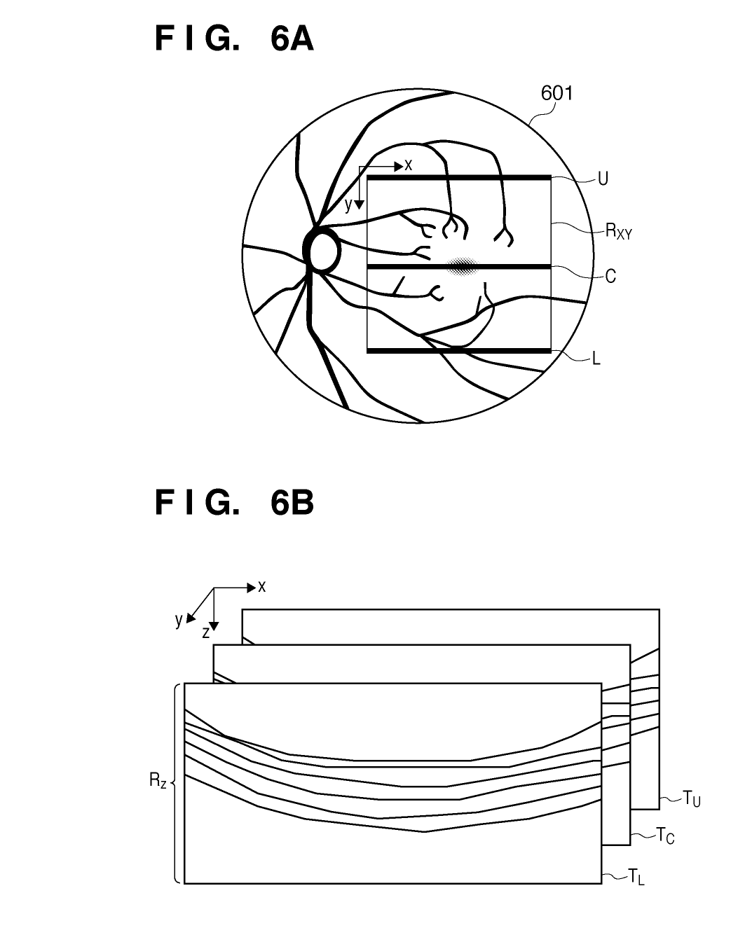

FIG. 6A is a diagram showing the tomogram acquisition positions P in the measurement area R_{XY}. Here, 601 denotes the fundus image, and R_{XY} in the fundus image 601 denotes the two-dimensional measurement area. Also, C in the measurement area R_{XY} denotes a line segment (hereinafter, referred to as the center portion) that passes through the center of the measurement area R_{XY} and is parallel to the x axis in the figure, U denotes the upper side (hereinafter, referred to as the upper edge portion) of the measurement area R_{XY}, and L denotes the lower side (hereinafter, referred to as the lower edge portion) of the measurement area R_{XY}. In the present embodiment, three positions at the center portion C, upper edge portion U, and lower edge portion L of the measurement area R_{XY} are applied as the positions indicating the center portion and two edge portions of the measurement area R_{XY}. These positions correspond to FIG. 13A (1302 to 1304). When acquiring tomograms as described later, the area of the measurement area R_{Z} in the z axis direction of these positions at the center portion C, the upper edge portion U, and the lower edge portion L are scanned to acquire the tomograms. However, as previously mentioned, the positions of the center and edge portions of the measurement area R_{XY} are not limited to those described above, and these positions may be positions such as those shown in FIGS. 13B to 13F. The tomogram acquisition positions P (the center portion C, upper edge portion U, and lower edge portion L) set in this way are transmitted to a tomogram acquisition unit 103.

In step S303, a moving amount setting unit 109 acquires, from the instruction acquisition unit 100, instruction information from the operator that is for manually setting a measurement position in the depth direction of the retina. This instruction is input by the operator with use of a user interface that is not shown. As one example of the instruction for setting the measurement position, the moving amount setting unit 109 acquires a moving amount (hereinafter, referred to as a depth-direction moving amount D) by which the measurement position is to be moved in the depth direction (z axis direction). The depth-direction moving amount D that was set is then transmitted to the tomogram acquisition unit 103.

In step S303, the tomogram acquisition unit 103 captures tomograms of the test-subject eye based on the tomogram acquisition positions P acquired from the tomogram acquisition position setting unit 102 and the depth-direction moving amount D acquired from the moving amount setting unit 109.

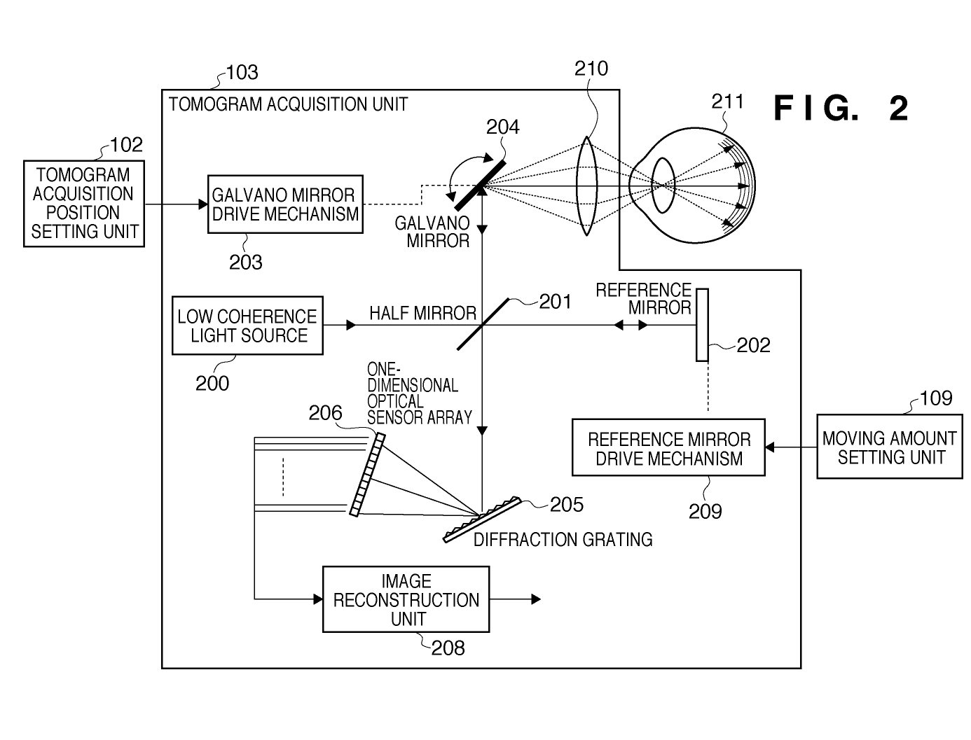

In the present embodiment, the tomogram acquisition unit 103 employs a Fourier domain method of OCT. FIG. 2 shows an example of a functional configuration and device configuration of the tomogram acquisition unit 103. The tomogram acquisition unit 103 controls a galvano mirror drive mechanism 203 in accordance with the tomogram acquisition positions P, and thus drives a galvano mirror 204. The galvano mirror drive mechanism 203 drives the galvano mirror 204 so as to scan signal light in the main scanning direction and the sub scanning direction (the x axis direction and the y axis direction in FIG. 6A). Here, since tomograms are captured in real-time at the three positions at the center portion C, the upper edge portion U, and the lower edge portion L in FIG. 6A, control is performed so that these three positions are scanned simultaneously in one instance of main scanning. Specifically, control is performed so that by switching the scan position in the sub scanning direction between the center portion C, the upper edge portion U, and the lower edge portion L at high speed, scanning in the main scanning direction is performed at a sampling interval that is ⅓ of that in the case of performing main scanning with the sub scanning position being fixed. Also, the tomogram acquisition unit 103 controls a reference mirror drive mechanism 209 in accordance with the depth-direction moving amount D, thus driving a reference mirror 202.

A light beam from a low coherence light source 200 is divided by a half mirror 201 into signal light that travels to a measurement object 211 via an objective lens 210 and reference light that travels to the reference mirror 202. Next, interfering light is generated by superposing the signal light and reference light that have been reflected by the measurement object 211 and the reference mirror 202 respectively. This interfering light is separated into wavelength components having wavelengths from λ1 to λn by a diffraction grating 205, and the wavelength components are detected by a one-dimensional optical sensor array 206. The one-dimensional optical sensor array 206 is configured by optical sensors that output detection signals indicating the light intensity of the detected wavelength components to an image reconstruction unit 208.

Based on the detection signals regarding the wavelength components of the interfering light that were output from the one-dimensional optical sensor array 206, the image reconstruction unit 208 obtains the relationship between wavelength and light intensity for the interfering light, that is to say, the light intensity distribution (wavelength spectrum) of the interfering light. Inverse Fourier transform is performed on the obtained interfering light wavelength spectrum, and a tomogram of the retina is reconstructed.

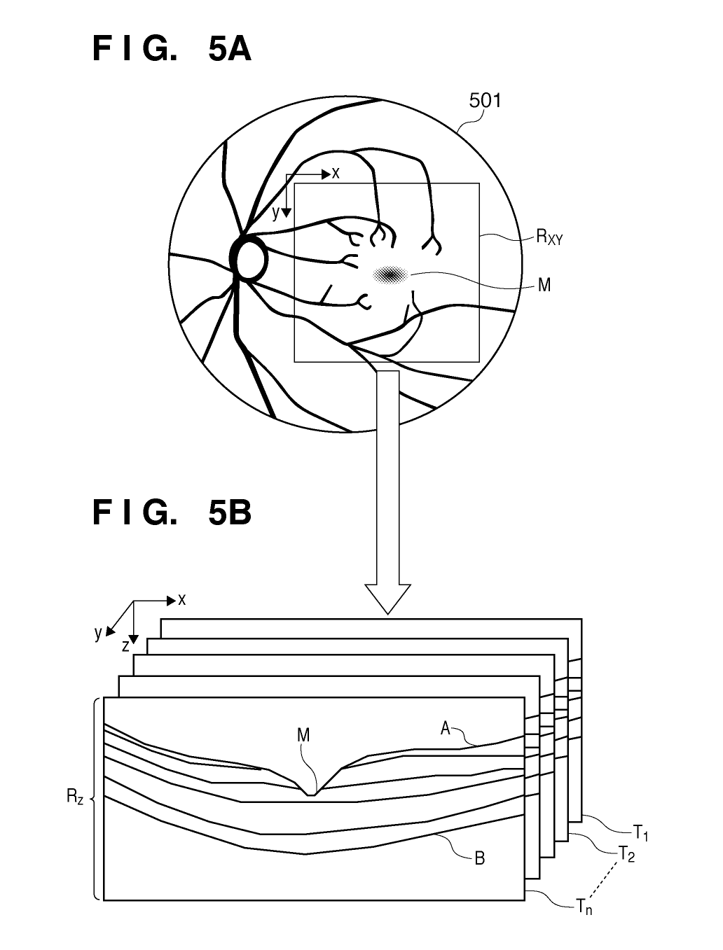

FIG. 6B is a diagram showing tomograms acquired at the center portion C, the upper edge portion U, and the lower edge portion L. Here, R_{Z} denotes the one-dimensional measurement area in the z axis direction, likewise to FIG. 5B. The measurement area R_{Z} is a range in the depth direction of the tomogram, and is determined based on the position to which the reference mirror 202 was moved under control. In FIG. 6B, T_{C} denotes a tomogram corresponding to the center portion C in FIG. 6A (hereinafter, referred to as the center portion tomogram), T_{U} denotes a tomogram corresponding to the upper edge portion U in FIG. 6A (hereinafter, referred to as the upper edge portion tomogram), and T_{L} denotes a tomogram corresponding to the lower edge portion L in FIG. 6A (hereinafter, referred to as the lower edge portion tomogram). The image data of the captured tomograms is transmitted to a storage unit 104.

Next, in step S305, a display method setting unit 105 acquires the tomogram image data stored in the storage unit 104, and sets a display method for simultaneously displaying the tomogram image data inline (defined as a display method 1).

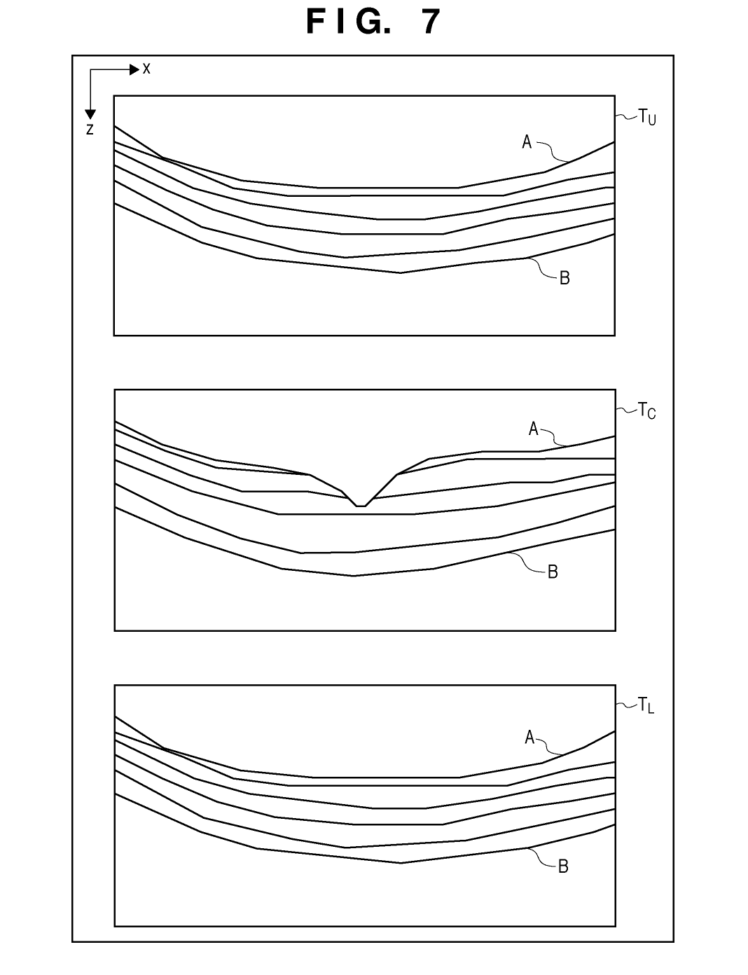

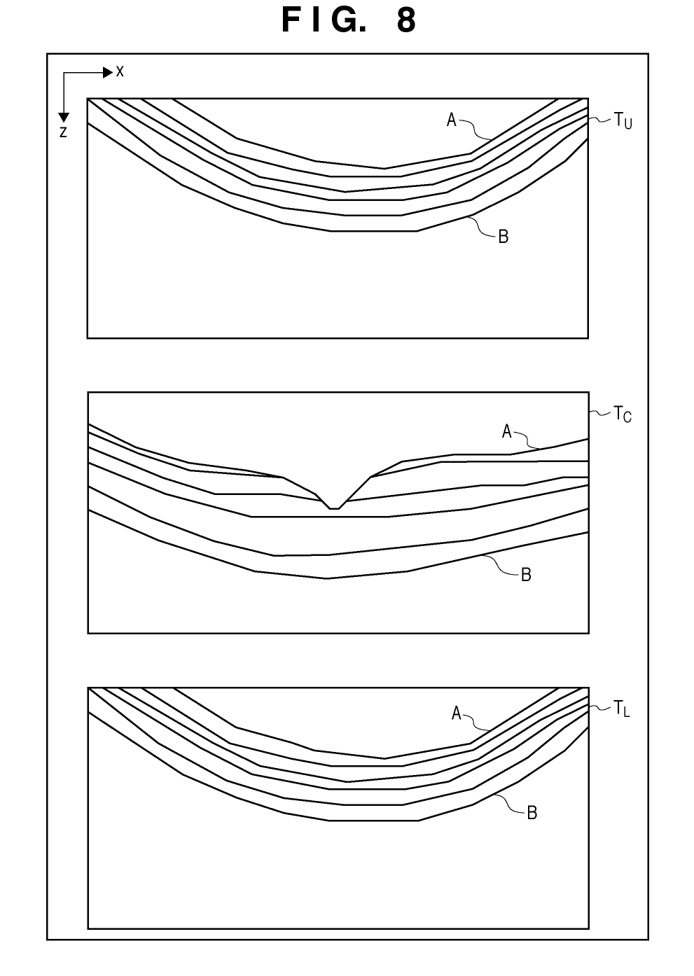

FIG. 7 shows an example of display according to the display method 1. Here, T_{U} denotes the upper edge portion tomogram, T_{C} denotes the center portion tomogram, and T_{L} denotes the lower edge portion tomogram. Also, likewise to FIG. 5B, A denotes the inner limiting membrane in the tomograms, and B denotes the retinal pigment epithelium boundary. As shown in FIG. 7, the method applied in the present embodiment is a method in which the upper edge portion tomogram T_{U}, the center portion tomogram T_{C}, and the lower edge portion tomogram T_{L} are displayed inline from the top in the stated order. However, the tomogram display method is not limited to this, and may be any method that enables the tomograms to be checked simultaneously in an inline condition. For example, these tomograms may be displayed lined up horizontally or diagonally. Also, FIG. 8 shows an example of display according to the display method 1 in the case where retina layers have been cut off. Likewise to FIG. 7, T_{U}, T_{C}, T_{L}, A, and B in FIG. 8 denote the upper edge portion tomogram, the center portion tomogram, the lower edge portion tomogram, the inner limiting membrane, and the retinal pigment epithelium boundary. In FIG. 8, the inner limiting membrane A has been cut off at the upper side in the upper edge portion tomogram T_{U} and the lower edge portion tomogram T_{L}. The direction in which the tomograms are lined up is the same as in FIG. 7.

In this way, by displaying the tomograms at the center and the edge portions inline, the user can check whether a retina layer has been cut off in a tomogram, thus enabling the user to determine whether a retina layer will be cut off in three-dimensional data obtained after imaging. The data regarding the display method 1 that has been set and the tomogram image data that is to be displayed are transmitted to a display unit 106.

Next, the processing from steps S306 to S308 is processing in which cutoff of a retina layer is detected in a tomogram, and a warning is displayed by causing the display form of the tomogram in which cutoff was detected to be different from that of the other tomograms. First, in step S306, a retina layer extraction unit 107 acquires the tomogram image data stored in the storage unit 104, and extracts retina layers from each of the tomograms through image analysis.

In this step, two layers, namely the inner limiting membrane A and the retinal pigment epithelium boundary B in FIG. 7, are extracted as the retina layers. The inner limiting membrane A is a boundary sandwiched between a vitreous body area on the top side that is extracted as a low luminance area in the image and a nerve fiber layer on the bottom side that is extracted as a high luminance area, and therefore the inner limiting membrane A has the property that the luminance gradient is high. In view of this, in the present embodiment, in one A-scan line, pixels of interest are scanned in order in the z axis positive direction beginning from the upper edge of the image, and a position at which the gradient in the image in the vicinity of the pixel of interest exceeds a certain threshold T_{A} is detected, thus extracting the pixel at the detected position as a pixel corresponding to the inner limiting membrane A. This is repeated for all of the A-scan lines, thus extracting the inner limiting membrane from the tomogram.

Also, an area (the retinal pigment epithelium) sandwiched between the retinal pigment epithelium boundary B and the photoreceptor inner segment/outer segment junction (IS/OS) that is a boundary one level above is extracted as a particularly high luminance area within the retina layers. Since the area above the IS/OS has a relatively low luminance in comparison to this area, the IS/OS has the property that the luminance gradient in the image is high. In view of this, in the present embodiment, in one A-scan line, pixels of interest are scanned in the z axis positive direction using the extracted position of the inner limiting membrane A as the origin, and a position at which the gradient in the image in the vicinity of the pixel of interest exceeds a certain threshold T_{I} is detected, thus extracting the pixel at the detected position as a pixel corresponding to the IS/OS. This is repeated for all of the A-scan lines, thus extracting the IS/OS layer from the tomogram.

Then, in one A-scan line, pixels of interest are furthermore scanned in the z axis positive direction using the IS/OS as the origin, and a position at which the luminance value is lower than a certain threshold T_{B} is detected, thus extracting the pixel at the detected position as a pixel corresponding to the retinal pigment epithelium boundary B. This is repeated for all of the A-scan lines, thus extracting the retinal pigment epithelium boundary B layer. Then, the tomogram image data and extracted retina layer data (three pieces of boundary data for the inner limiting membrane, the IS/OS, and the retinal pigment epithelium boundary) are transmitted to a retina layer cutoff detection unit 108.

In step S307, the retina layer cutoff detection unit 108 detects cutoff of a retina layer based on the tomogram image data and the retina layer data acquired from the retina layer extraction unit 107, and generates retina layer data in which cutoff of a retina layer has been detected. This data is defined as retina layer cutoff detection data. If cutoff, that is to say, the fact that a retina layer protrudes outside the measurement range in the depth direction has been detected, a flag indicating the a retina layer has been cut off is set to True, and if cutoff has not been detected, the flag is set to False. This flag is defined as a cutoff detection flag E. Then, if the cutoff detection flag E is True, the value of the cutoff detection flag E and the retina layer cutoff detection data are transmitted to the storage unit 104, and the procedure moves to step S308. If the cutoff detection flag E is False, only the value of the cutoff detection flag E is transmitted to the storage unit 104, and the procedure moves to step S312.

In this step, cutoff of a retina layer is detected by the following method. First is a description of a method for detecting cutoff of the inner limiting membrane A in the tomogram shown in FIG. 8. In one A-scan line, the point on the inner limiting membrane A that was detected in step S306 is set as p_{A}. Here, a certain area (for example, approximately three pixels) above (in the z axis negative direction) the point p_{A} is defined as a reference area X. Then, if the luminance values in the reference area X fall within a certain range based on the luminance values of the vitreous body area originally existing above the inner limiting membrane A, a determination is made that cutoff has not occurred, and if these luminance values do not fall within this range, a determination is made that cutoff has occurred. This condition expression is as shown below.

V_{Corpus}−T_{A}≦V_{X}≦V_{Corpus}+T_{A} (1)

In Expression (1), V_{X} is the average luminance value in the reference area X, V_{Corpus} is the average luminance value of the vitreous body area, and T_{A} is a positive constant indicating a certain range of luminance values. In this way, if Expression (1) is not satisfied, the point detected as the point p_{A} is not adjacent to the vitreous body area, and therefore it is considered that the inner limiting membrane A does not appear on the A-scan line, and has been cut off at the upper side of the tomogram. Also, if a pixel does not exist above the point p_{A}, the point p_{A} is located at the upper side of the tomogram, and therefore a determination is made that cutoff has occurred.

Next is a description of a method for detecting cutoff of the retinal pigment epithelium boundary B. In one A-scan line, the point on the retinal pigment epithelium boundary B that was detected in step S306 is set as p_{B}, and the detected point on the IS/OS is set as p_{I}. Here, a region sandwiched between the points p_{I} and p_{B} is defined as a reference region Y. Then, if the luminance values in the reference area Y fall within a certain range based on the luminance values of the retinal pigment epithelium area originally sandwiched between the IS/OS and the retinal pigment epithelium boundary, a determination is made that cutoff has not occurred, and if these luminance values do not fall within this range, a determination is made that cutoff has occurred. This condition expression is as shown below.

V_{RPE}−T_{B}≦V_{Y}≦V_{RPE}+T_{B} (2)

In Expression (2), V_{Y} is the average luminance value of the reference area Y, V_{RPE} is the average luminance value of the retinal pigment epithelium, and T_{B} is a positive constant indicating a certain range of luminance values. In this way, if Expression (2) is not satisfied, the points detected as the points p_{B} and p_{I} are not adjacent to the retinal pigment epithelium area, and therefore it is considered that the retinal pigment epithelium does not appear on the A-scan line, and has been cut off at the lower side of the tomogram. Also, even if Expression (2) is satisfied, if a pixel does not exist below the point p_{B}, the point p_{B} is located at the lower side of the tomogram, and therefore a determination is made that cutoff has occurred.

Retina layer data (for the inner limiting membrane A or the retinal pigment epithelium boundary B) for which a determination regarding cutoff has been made for each A-scan line as described above and the tomogram image data is combined, and the combined data is the retina layer cutoff detection data.

Next, in step S308, the display method setting unit 105 acquires, from the storage unit 104, data indicating that the cutoff detection flag E is True and the retina layer cutoff detection data, and sets the display method to a method for displaying a warning (defined as a display method 2). As will be described below, with the display method 2, the display form of a tomogram for which it has been detected that a retina layer protrudes outside the measurement range in the depth direction is caused to be different from the display form of the other tomograms (the display form of the tomograms for which the retina layers do not protrude outside the measurement range).

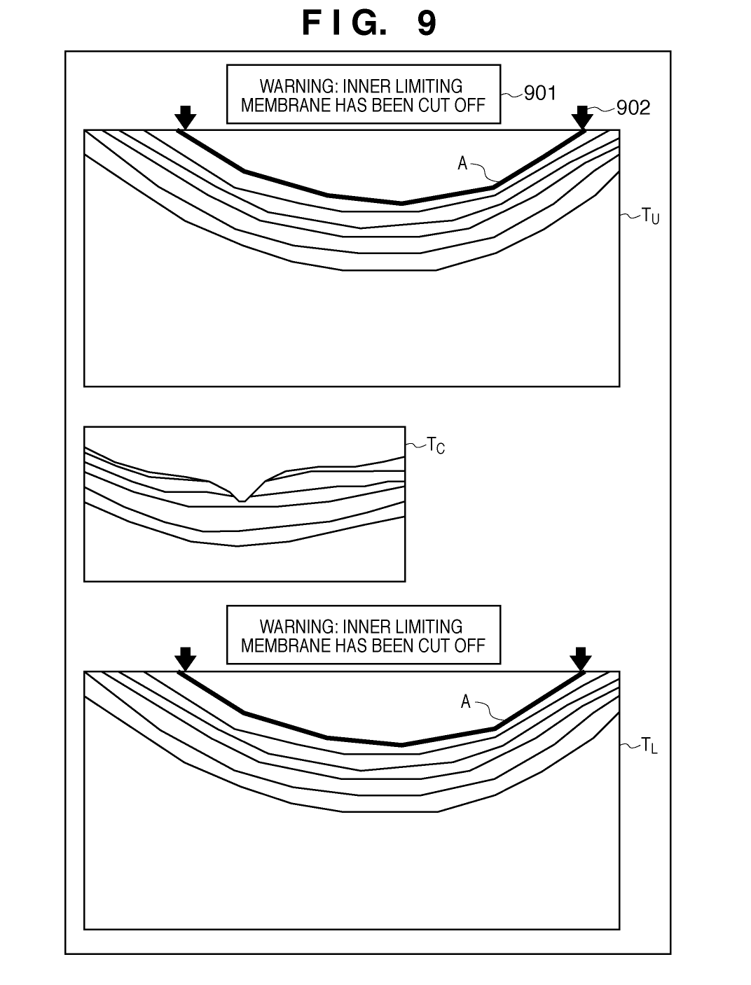

FIG. 9 is a diagram showing warning display that is performed in the case where the inner limiting membrane has been cut off, as an example of display according to the display method 2. This display method is applied in the case where the retina layer cutoff detection data is inner limiting membrane data. The display method 2 in FIG. 9 corresponds to a display in which portions of the display method 1 in FIG. 8 have been changed for warning display. In FIG. 9, T_{U} denotes the upper edge portion tomogram, T_{C} denotes the center portion tomogram, and T_{L} denotes the lower edge portion tomogram. Also, 901 denotes a warning display in which the fact that the inner limiting membrane has been cut off is communicated using a sentence, and 902 denotes arrows indicating the positions of end points of the inner limiting membrane at which cutoff has occurred. Also, the inner limiting membranes A that have been cut off in the tomograms are indicated using bold lines for emphasis. In FIG. 9, the tomograms T_{U} and T_{L} in which the inner limiting membrane has been cut off are shown at an increased size, and the tomogram T_{C} in which cutoff has not occurred is shown at a decreased size.

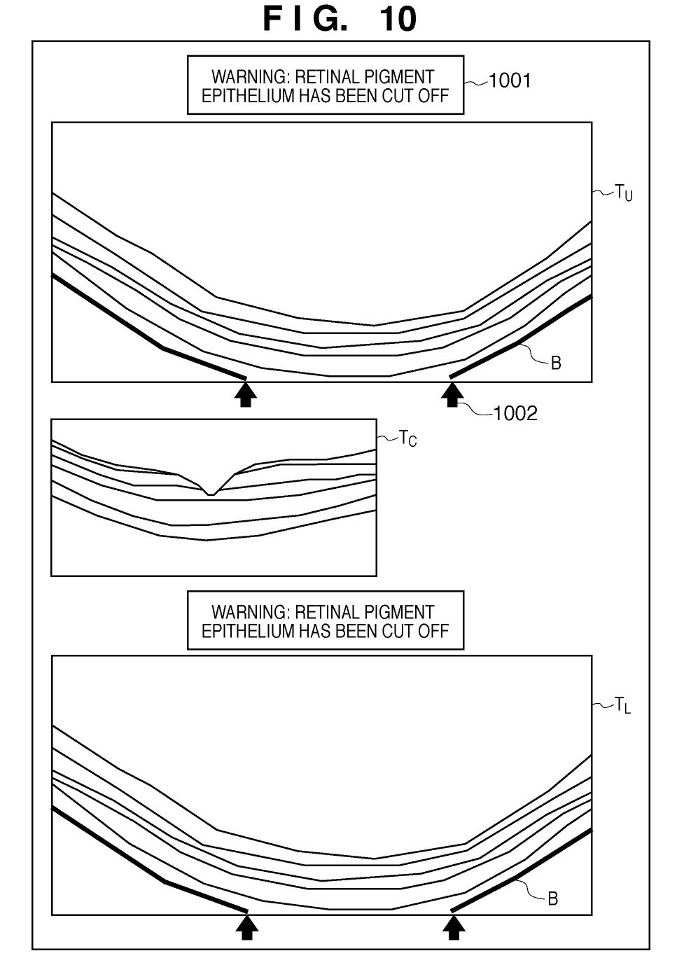

Also, FIG. 10 is a diagram showing a warning display that is displayed in a case where the retinal pigment epithelium boundary has been cut off, as an example of a display according to the display method 2. This display method is applied in the case where the retina layer cutoff detection data is retinal pigment epithelium boundary data. In FIG. 10, T_{U} denotes the upper edge portion tomogram, T_{C} denotes the center portion tomogram, and T_{L} denotes the lower edge portion tomogram. Also, 1001 denotes a warning display in which the fact that the retinal pigment epithelium boundary has been cut off is communicated using a sentence, and 1002 denotes arrows indicating the positions of end points of the retinal pigment epithelium boundary at which cutoff has occurred. Also, the retinal pigment epithelium boundaries B that have been cut off in the tomograms are indicated using bold lines for emphasis. In FIG. 10, the tomograms T_{U} and T_{L} in which the retinal pigment epithelium boundary has been cut off are shown in at an increased size, and the tomogram T_{C} in which cutoff has not occurred is shown at a decreased size.

In this way, the fact that a retina layer has been cut off is displayed by displaying a sentence, displaying the cutoff places, displaying the layer in an emphasized manner, and displaying the tomograms at an increased size, thus helping make the observer aware that a retina layer has been cut off. Then, the data regarding the display method 2 is transmitted to the display unit 106. Thereafter, in step S309, the display unit 106 acquires the data regarding the display method 2 from the display method setting unit 105, and performs display control so as to display the acquired data on a monitor that is not shown.

Next, in step S310, if the cutoff detection flag E is True, the moving amount setting unit 109 determines whether input from the observer that is for instructing automatic adjustment of the measuring depth to be performed has been acquired from the instruction acquisition unit 100. This instruction is input by the operator using a user interface that is not shown. Here, if the moving amount setting unit 109 has acquired input instructing automatic adjustment to be performed, the procedure moves to step S311. If such input has not been acquired, the procedure returns to step S303.

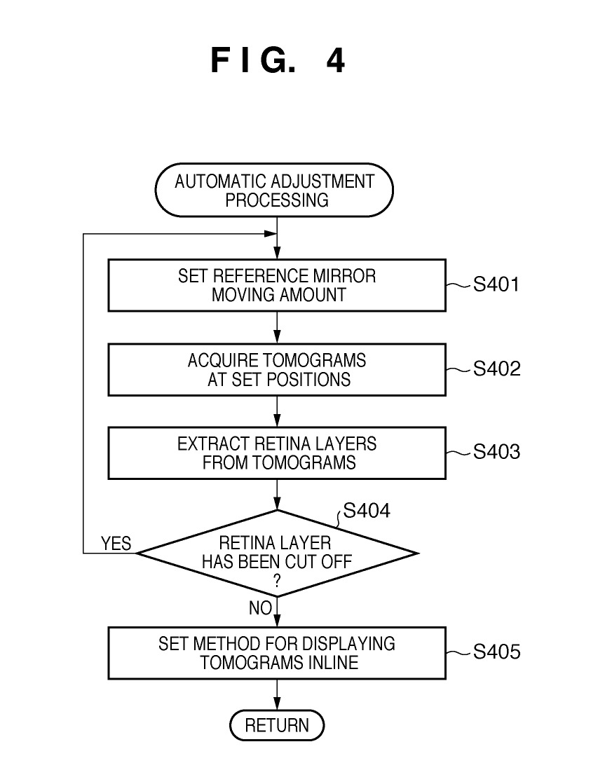

In step S311, the moving amount setting unit 109 sets a moving amount for automatically adjusting the measuring depth in the depth direction with respect to the retina, based on the retina layer cutoff detection data acquired in step S310. Next, the tomogram acquisition unit 103 acquires tomogram image data based on the moving amount that was set. Thereafter, the display method setting unit 105 sets the display method for the acquired tomograms to the display method 1. Then, the data regarding the display method 1 that has been set is transmitted to the display unit 104. Details of the processing in this step will be described later with reference to the flowchart shown in FIG. 4.

In step S312, the display unit 106 acquires the data regarding the display method 1 from the display method setting unit 105, and performs display control so as to display the tomograms on the monitor that is not shown. Then, in step S313 the instruction acquisition unit 100 acquires, from the outside, an instruction indicating whether to end the processing for analyzing and displaying tomograms that is performed by the tomography apparatus 10. This instruction is input by the operator using a user interface that is not shown. If the acquired instruction is not for ending processing, but rather designates a place of interest on the fundus image, the procedure returns to step S311. If an instruction for ending processing has been acquired, the tomography apparatus 10 ends such processing. Note that if a user instruction for changing the measuring range has been acquired here, the procedure returns from step S313 to S301.

In this way, the processing shown in FIG. 3 ends, and imaging for diagnosis is executed after the measuring depth has been appropriately set, thus enabling the photographer to more accurately obtain appropriate tomograms.

Next is a description of the processing for automatic measuring depth adjustment in step S311 with reference to FIG. 4.

In step S401, the moving amount setting unit 109 acquires the retina layer cutoff detection data from the retina layer cutoff detection unit 108, analyzes it, and sets a necessary moving amount (defined as a depth-direction moving amount D′). When capturing tomograms, the reference mirror 202 moves by merely the depth-direction moving amount D, and therefore tomograms are acquired in the measurement area R_{Z}. Also, when the depth-direction moving amount D′ is set, the moving start position of the depth-direction moving amount D by which the reference mirror 202 moves when capturing tomograms is offset by merely D′, and thus the measurement area R_{Z} is offset by merely D′. In this step, if the retina layer cutoff detection data is data indicating cutoff of the inner limiting membrane, the upper side of the retina layers has been cut off, and therefore the measurement area R_{Z} is moved in the z axis negative direction (the depth-direction moving amount D′ is a negative value). On the other hand, if the retina layer cutoff detection data is data indicating the retinal pigment epithelium boundary, the lower side of the retina layers has been cut off, and therefore the measurement area R_{Z} is moved in the z axis positive direction (the depth-direction moving amount D′ is a positive value).

First is a description of a method for setting the depth-direction moving amount D′ in the case where the inner limiting membrane has been cut off (the case where the depth-direction moving amount D′ is a negative value). In the present embodiment, a configuration is possible in which d_{C} is a positive constant, and setting is performed such that the depth-direction moving amount D′ is −d_{C}, and a configuration is possible in which a distance d_{X} (positive value) necessary for preventing cutoff of a retina layer is calculated, and setting is performed such that the depth-direction moving amount D′ is −d_{X}. A method for setting d_{X} is described below.

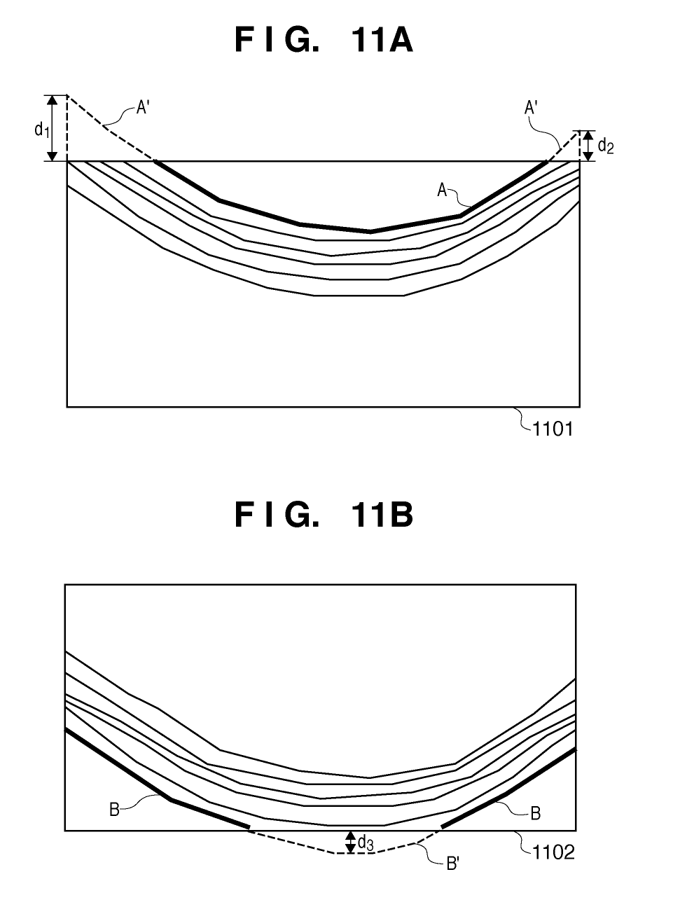

FIG. 11A is a diagram showing the positional relationship between a tomogram and an estimated line of the inner limiting membrane that has been cut off. Here, 1101 denotes an edge portion tomogram (upper edge portion tomogram or lower edge portion tomogram), and A denotes the inner limiting membrane. Also, A′ denotes an estimated line of the inner limiting membrane that has been cut off, which has been estimated by extrapolating the outline of the inner limiting membrane A. Furthermore, d₁ denotes the distance between the estimated line A′ and the left edge of the upper side of the tomogram, and d₂ denotes the distance between the estimated line A′ and the right edge of the upper side of the tomogram.

As a method of estimating the estimated line A′, using the fact that the three-dimensional shape of the retina can be approximated to an ellipsoidal body, a curve obtained by, for example, fitting an ellipsoidal shape to the inner limiting membrane A that has been detected is applied as the estimated line A′. The method is not limited to this, and another estimation method that takes the shape of the retina into consideration may be used.

Then, whichever of the distance d₁ and the distance d₂ has the largest value is applied as the distance d_{X}. In the example in FIG. 11A, d₁>d₂, and therefore d_{X}=d₁. Accordingly, the distance from the upper side of the tomogram to the position on the estimated line A′ that is the farthest away from the upper side of the tomogram can be set as the distance d_{X}. Therefore, moving the measurement area R_{Z} by merely distance d_{X} in the z axis negative direction enables causing the entirety of the inner limiting membrane A to fit in the tomogram.

Next is a description of a method for setting the depth-direction moving amount D′ in the case where the retinal pigment epithelium boundary has been cut off. In this case, contrary to the symbols in the case where the inner limiting membrane has been cut off, setting may be performed such that D′ is d_{C}, or such that D′ is d_{X}. A method for setting d_{X} is described below.

FIG. 11B is a diagram showing the positional relationship between a tomogram and an estimated line of the retinal pigment epithelium boundary that has been cut off. Here, 1102 denotes an edge portion tomogram (upper edge portion tomogram or lower edge portion tomogram), and B denotes the retinal pigment epithelium boundary. Also, B′ denotes an estimated line obtained by extrapolating the outline of the retinal pigment epithelium boundary that has been cut off, and d₃ denotes the distance between the lower side of the tomogram and the position on the estimated line B′ having the largest z coordinate. Since the retinal pigment epithelium boundary is also part of the retina, the estimated line B′ can also be obtained using the same method as with the estimated line A′. Then, setting the distance d_{X} to d₃ and moving the measurement area R_{Z} by merely d_{X} in the z axis positive direction enables causing the entirety of the retinal pigment epithelium boundary B to fit in the tomogram. The value of the depth-direction moving amount D′ set in this way is transmitted to the tomogram acquisition unit 103.

In step S402, the tomogram acquisition unit 103 captures tomograms of the test-subject eye based on the depth-direction moving amount D′ acquired from the moving amount setting unit 109 and the tomogram acquisition positions P set by the tomogram acquisition position setting unit 102 in step S302. Details of this processing have been omitted due to being the same as in step S303. The image data of the captured tomograms is transmitted to the storage unit 104.

In step S403, the retina layer extraction unit 107 acquires the tomogram image data stored in the storage unit 104, and extracts the retina layers through image analysis. Details of this processing have been omitted due to being the same as in step S306. The tomogram image data and the retina layer data that has been extracted are transmitted to the retina layer cutoff detection unit 108. Then, in step S404 the retina layer cutoff detection unit 108 detects cutoff of a retina layer based on the tomogram image data and extracted retina layer data that have been acquired from the retina layer extraction unit 107. Details of this processing have been omitted due to being the same as in step S307. If the cutoff detection flag E is True, the value of the cutoff detection flag E and the retina layer cutoff detection data are transmitted to the storage unit 104, and the procedure moves to step S401. If the cutoff detection flag E is False, only the value of the cutoff detection flag E is transmitted to the storage unit 104, and the procedure moves to step S405.

In step S405, the display method setting unit 105 acquires the tomogram image data and the value indicating that the cutoff detection flag E is False that are stored in the storage unit 104, and sets a display method for simultaneously displaying the tomogram image data inline, that is to say, sets the display method 1. Details of this processing have been omitted due to being the same as in step S304. The data regarding the display method 1 that has been set and the tomogram image data that is to be displayed are transmitted to the display unit 106.

According to the above-described procedure, the measuring depth is automatically adjusted. It should be noted that if the depth-direction moving amount D′ has been set to −d_{C} (the inner limiting membrane has been cut off) in step S401, tomograms are acquired in step S402 after having moved the reference mirror by merely a certain amount. Accordingly, the procedure for performing this processing and then checking for cutoff of a retina layer in step S404 is performed repeatedly (steps S401 to S404), and then the procedure moves to the next processing when cutoff of a retina layer is no longer detected.

On the other hand, if the depth-direction moving amount D′ has been set to −d_{X} in step S401, tomograms are captured in step S402 after having moved the reference mirror merely a distance necessary to prevent cutoff from occurring. Accordingly, after cutoff of a retina layer has been checked for once in step S404, the procedure moves to the next processing. Alternatively, in this case, the reference mirror is moved merely the distance that is necessary, and therefore the check processing performed through steps S403 and S404 may be omitted.

According to the configuration described above, since the three-dimensional shape of the retina can be approximated to an ellipsoidal body, it is possible to observe tomograms at the center and edge portions of the measurement area R_{XY} for a test-subject eye in real-time, which enables making a determination during observation of the test-subject eye whether all of the retina layers will fit in three-dimensional data that will be obtained after imaging. Then, if cutoff of a retina layer has been detected, a warning to that effect is presented, thereby helping making the photographer aware that a retina layer has been cut off. Furthermore, if cutoff of a retina layer has been detected, in accordance with an instruction from the photographer, the condition of the cutoff of the retina layer is analyzed, and the measuring depth in the depth direction of the retina is automatically adjusted so that the retina layer is not cutoff in the tomogram, thereby mitigating the burden on the photographer for making positional adjustments, and furthermore preventing imaging mistakes.

On the other hand, if cutoff of a retina layer has not been detected, and furthermore the photographer has not given an automatic adjustment instruction, the measuring depth can be acquired by manual input. According to this configuration, the photographer can manually position the measuring depth (position the reference mirror 202) while observing warning displays that indicate cutoff of a retina layer. Here, the results of positioning the reference mirror 202 are displayed according to the display method 2 in real-time, and display according to the display method 1 is performed when the cutoff of a retina layer has been resolved.

Also, by omitting the retina layer extraction unit 107 and the retina layer cutoff detection unit 108 in FIG. 1, and also the processing regarding the display method 2 performed in the display method setting unit 105, it is possible to achieve a configuration in which only the display method 1 is applied in the display method setting unit 105, and tomograms are displayed by the display unit 106 in real-time. In this case, cutoff of a retina layer is not detected, and therefore only a manual measuring depth instruction from the photographer is input to the moving amount setting unit 109. Accordingly, the photographer can manually align the measuring depth while observing unadjusted tomograms at the center position and two edge portion positions of the measurement area R_{XY} in real-time. In this case, S303 to S305 in FIG. 3 are executed repeatedly.

Additionally, in the case of performing automatic adjustment, there is no need for the user to check the tomograms. Accordingly, the display method in the display method setting unit 105 may be set to a method of displaying only one tomogram at the center portion. In this way, a configuration is possible in which the tomograms at the two edge portions are not displayed, nor is a warning regarding cutoff of a retina layer displayed, and the moving amount setting unit 109 automatically adjusts the measuring depth if a retina layer has been cut off. In this case, the condition of the tomograms at the two edge portions is not presented to the photographer, and therefore it is possible for the photographer to acquire three-dimensional data free from imaging mistakes based on a position that has been automatically adjusted by a computer, without being aware of the condition of the tomograms at the edge portions. With this configuration, if cutoff of a retina layer has occurred, the photographer may be notified that cutoff has occurred using some sort of notification means other than presentation of tomograms (for example, an audio notification), an instruction may be then acquired from the photographer, and then automatic adjustment may be performed, or alternatively, automatic adjustment may be performed without notifying the photographer.

### Other Embodiments

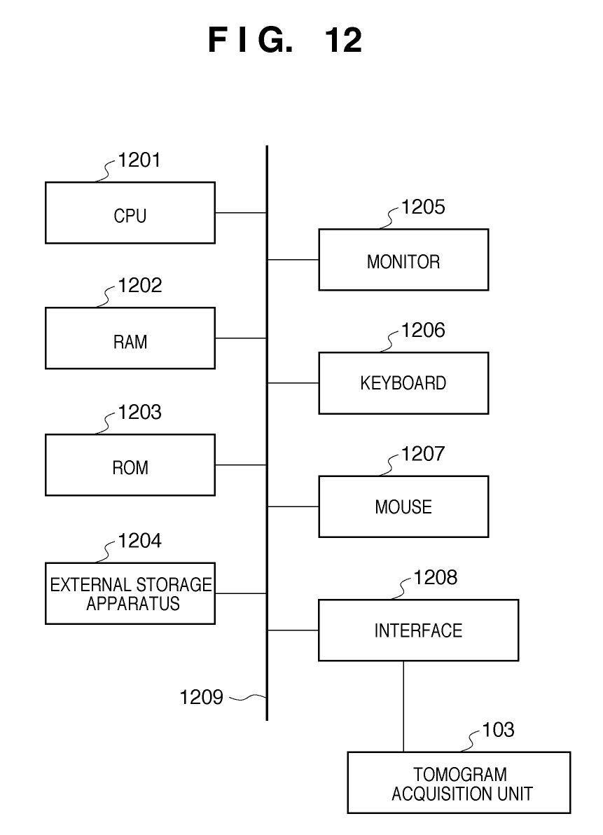

In the above embodiment, the present invention is realized as an imaging apparatus. However, the embodiments of the present invention are not limited to only an imaging apparatus. In the present embodiment, a configuration is described in which the present invention is realized as software running on a computer. FIG. 12 is a diagram showing a basic configuration of a computer for realizing the functions of the various units of the tomography apparatus 10 with software.

A CPU 1201 performs overall control of the computer with use of computer programs and data that are stored in a RAM 1202 and a ROM 1203. Also, the CPU 1201 realizes the functions of the various units by controlling the execution of the software corresponding to the various units of the tomography apparatus 10. The RAM 1202 is provided with an area for temporarily storing computer programs and data that have been loaded from an external storage apparatus 1204, and also a work area necessary for the CPU 1201 to perform various processing. The functions of the storage unit 104 are realized by the RAM 1202. The ROM 1203 generally stores a computer BIOS, setting data, and the like. The external storage apparatus 1204 is an apparatus that functions as a high-capacity information storage apparatus such as a hard disk drive, and the external storage apparatus 1204 stores an operating system, the computer programs executed by the CUP 1201, and the like. Also, the external storage apparatus 1204 stores the information made known in the description of the present embodiment, and such information is loaded to the RAM 1202 as necessary. A monitor 1205 is configured by a liquid crystal display or the like. The monitor 1205 can, for example, display the content output by the display unit 106. A keyboard 1206 and a mouse 1207 are input devices, and can be used by the operator to give various instructions to the tomography apparatus 10. An interface 1208 is an interface for exchanging data with the tomogram acquisition unit 103. Note that an interface configured by IEEE 1394, USB, an Ethernet (registered trademark) port, or the like may be provided for exchanging various data with external devices. Data acquired via the interface 1208 is loaded to the RAM 1202. The constituent elements described above are connected to each other via a bus 1209.

Note that the functions of the various units of the tomography apparatus 10 in the present embodiment are realized by the CPU 1201 executing the computer programs for realizing the functions of the various units, and performing overall control of the computer. Also, it is assumed that program code corresponding to the flowcharts described in the above embodiment has been, for example, loaded to the RAM 1202 from the external storage apparatus 1204.

As described above, according to the above embodiments, tomograms at the center position and edge portion positions in a measurement area of a test-subject eye are displayed inline in real-time, thereby enabling making a determination when observing a test-subject eye whether all of the retina layers will fit in three-dimensional data that will be captured thereafter. Alternatively, the imaging area in the depth direction is automatically adjusted so that the retina layers do not protrude outside the imaging area in the depth direction in the tomograms. This enables preventing an imaging mistake in which a retina layer has been cut off in a tomogram obtained after capturing the three-dimensional data.

Note that although the description in the embodiments of the present invention as described above give an example of a preferable image processing apparatus according to the present invention, the present invention is not limited to this.

According to the embodiments described above, in an imaging apparatus that employs optical coherence tomography, it is possible to easily and appropriately set, in a measurement area that has been set, an imaging position in the depth direction of a tomogram.

Aspects of the present invention can also be realized by a computer of a system or apparatus (or devices such as a CPU or MPU) that reads out and executes a program recorded on a memory device to perform the functions of the above-described embodiments, and by a method, the steps of which are performed by a computer of a system or apparatus by, for example, reading out and executing a program recorded on a memory device to perform the functions of the above-described embodiments. For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-186779, filed Aug. 11, 2009, which is hereby incorporated by reference herein in its entirety.

1. A control method for a tomography apparatus that captures a tomogram of a fundus through optical coherence tomography, the control method comprising:
a setting step of setting, on the fundus, a measurement area in which a tomogram is to be captured;
an acquisition step of acquiring tomograms with use of the optical coherence tomography at a plurality of predetermined positions in the measurement area, the number of the predetermined positions being smaller than that in a case of imaging for diagnosis; and
a display control step of displaying the tomograms acquired in the acquisition step inline on a screen of a display apparatus in real-time.

2. The control method according to claim 1, wherein in said display control step, the tomograms are displayed in same size.

3. The control method according to claim 1, further comprising:
an extraction step of extracting a retina layer from each of the tomograms acquired in the acquisition step; and
a detection step of, for each of the tomograms, detecting whether the retina layer extracted in the extraction step protrudes outside an imaging area in relation to a depth direction of the tomogram,
wherein in the display control step, a display form of each tomogram in which the retina layer has been detected as protruding is caused to be different from a display form of other tomograms.

4. A control method for a tomography apparatus that captures a tomogram of a fundus through optical coherence tomography, the control method comprising:
a setting step of setting, on the fundus, a measurement area in which a tomogram is to be captured;
an acquisition step of acquiring tomograms with use of the optical coherence tomography at a plurality of predetermined positions in the measurement area, the number of the predetermined positions being smaller than that in a case of imaging for diagnosis;
an extraction step of extracting a retina layer from each of the tomograms acquired in the acquisition step; and
an adjustment step of, based on positions in a depth direction in the tomograms of the retina layers extracted in the extraction step, adjusting a position of a reference mirror used in the optical coherence tomography so that the retina layers do not protrude outside an imaging area in the depth direction.

5. The control method according to claim 4,
wherein in the adjustment step,
a determination is made for each of the tomograms as to whether the extracted retina layer protrudes outside the imaging area in the depth direction,
in each case where a determination has been made that the retina layer protrudes, an amount by which the retina layer protrudes is estimated by extrapolating an outline of the retina layer, and
the position of the reference mirror is adjusted based on the estimated amounts of protrusion.

6. The control method according to claim 1, wherein the positions at which the tomograms are acquired are a center and an edge portion.

7. A tomography apparatus that captures a tomogram of a fundus through optical coherence tomography, comprising:
a setting unit that sets, on the fundus, a measurement area in which a tomogram is to be captured;
an acquisition unit that acquires tomograms with use of the optical coherence tomography at a plurality of predetermined positions in the measurement area, the number of the predetermined positions being smaller than that in a case of imaging for diagnosis; and
a display control unit that displays the tomograms acquired by the acquisition unit inline on a screen of a display apparatus in real-time.

8. A tomography apparatus that captures a tomogram of a fundus through optical coherence tomography, comprising:
a setting unit that sets, on the fundus, a measurement area in which a tomogram is to be captured;
an acquisition unit that acquires tomograms with use of the optical coherence tomography at a plurality of predetermined positions in the measurement area, the number of the predetermined positions being smaller than that in a case of imaging for diagnosis;
an extraction unit that extracts a retina layer from each of the tomograms acquired by the acquisition unit; and
an adjustment unit that, based on positions in a depth direction in the tomograms of the retina layers extracted by the extraction unit, adjusts a position of a reference mirror used in the optical coherence tomography so that the retina layers do not protrude outside an imaging area in the depth direction.

9. (canceled)

10. A non-transitory computer-readable storage medium that stores a computer program for causing a computer to execute the steps of the control method for a tomography apparatus according to claim 1.

11. A tomography apparatus for capturing a tomogram of a fundus by making a light returned from the fundus optically interfere with a light returned from a reference mirror, said apparatus comprising:
an acquisition unit that acquires a tomogram from an area in which a tomogram for diagnosis is captured, the number of the tomogram being smaller than the number of tomogram for diagnosis; and
a display control unit that live displays the tomogram obtained by the acquisition unit on a display apparatus.

12. A tomography apparatus for capturing a tomogram of a fundus by making a light returned from the fundus optically interfere with a light returned from a reference mirror, said apparatus comprising:
an acquisition unit that acquires a tomogram from an area of the fundus in which a tomogram for diagnosis are captured, the number of the tomogram being smaller than the number of tomogram for diagnosis;
a display control unit that displays the tomogram obtained by the acquisition unit on a display apparatus; and
a unit that changes an imaging position for the tomogram in accordance with an instruction by a user after the display control unit displays the tomogram.

13. A tomography apparatus for capturing a tomogram of a fundus by making a light returned from the fundus optically interfere with a light returned from a reference mirror, said apparatus comprising:
an acquisition unit that acquires a tomogram from an area in which a tomogram for diagnosis is captured, the number of the tomogram being smaller than the number of the tomogram for diagnosis; and
a display control unit that live displays the tomogram acquired by the acquisition unit on a display apparatus,
wherein the tomogram for diagnosis are captured after the display control unit displays the tomogram.

14. A tomography apparatus for capturing a tomogram of a fundus by making a light returned from the fundus optically interfere with a light returned from a reference mirror, said apparatus comprising:
an acquisition unit that acquires a tomogram from an area set in a two-dimensional image of the fundus, in which a tomogram for diagnosis are captured, the number of the tomogram being smaller than the number of tomogram for diagnosis; and
a display control unit that live displays the tomogram obtained by the acquisition unit on a display apparatus,
wherein the tomogram for diagnosis are captured after the display control unit displays the tomogram.

15. The apparatus according to claim 12, wherein the unit that changes the imaging position changes a position of the reference mirror.

16. The apparatus according to claim 11, wherein the acquisition unit acquires the tomogram based on a center position of the area.

17. A tomography apparatus comprising:
a low coherence light source;
an optical member which divaricates a low coherence light from the low coherence light source into a fundus of test-subject eye side and a reference mirror side;
a second optical member which scans the fundus of the test-subject eye by irradiating the fundus with the low coherence light divaricated by the optical member;
a reconstruction unit configured to obtain a tomogram by making the low coherence light returned from the fundus of the test-subject optically interfere with the low coherence light returned from the reference mirror;
a driving unit configured to drive the second optical member to obtain at least one tomogram from a two dimensional scan area on which the low coherence light is scanned; and
a display unit configured to live display the at least one tomogram,
wherein the drive unit obtains tomograms for diagnosis, from the two dimensional area, after the display unit displays the at least one tomogram.

18. A tomography apparatus for capturing a tomogram of a fundus by making a light returned from the fundus optically interfere with a light returned from a reference mirror, said apparatus comprising:
an acquisition unit that acquires, based on a capturing area for capturing a tomogram for diagnosis, a tomogram from an area smaller than the capturing area; and
a display control unit that live displays the tomogram obtained by the acquisition unit on a display apparatus.

19. A tomography apparatus for capturing a tomogram of a fundus by making a light returned from the fundus optically interfere with a light returned from a reference mirror, said apparatus comprising:
an acquisition unit that acquires, based on a capturing area for capturing a tomogram for diagnosis, a tomogram from an area smaller than the capturing area;
a display control unit that displays the tomogram obtained by the acquisition unit on a display apparatus; and
a unit that changes an imaging position for the tomogram in accordance with an instruction by a user after the display control unit displays the tomogram.

20. A tomography apparatus for capturing a tomogram of a fundus by making a light returned from the fundus optically interfere with a light returned from a reference mirror, said apparatus comprising:
an acquisition unit that acquires, based on a capturing area for capturing a tomogram for diagnosis, a tomogram from an area smaller than the capturing area; and
a display control unit that live displays the tomogram acquired by the acquisition unit on a display apparatus,
wherein the tomogram for diagnosis are captured after the display control unit displays the tomogram.

21. A tomography apparatus for capturing a tomogram of a fundus by making a light returned from the fundus optically interfere with a light returned from a reference mirror, said apparatus comprising:
an acquisition unit that acquires, based on a capturing area for capturing a tomogram for diagnosis, a tomogram from an area smaller than the capturing area, the capturing area being set in a two-dimensional image of the fundus; and
a display control unit that live displays the tomogram obtained by the acquisition unit on a display apparatus,
wherein the tomogram for diagnosis are captured after the display control unit displays the tomogram.

22. A tomography apparatus comprising:
a low coherence light source;
an optical member which divaricates a low coherence light from the low coherence light source into a fundus of test-subject eye side and a reference mirror side;
a second optical member which scans the fundus of the test-subject eye by irradiating the fundus with the low coherence light divaricated by the optical member;
a reconstruction unit configured to obtain a tomogram by making the low coherence light returned from the fundus of the test-subject optically interfere with the low coherence light returned from the reference mirror;
a driving unit configured to drive the second optical member to obtain, based on a two-dimensional scanning area which is scanned by the low coherence light, a tomogram from an area smaller than the two-dimensional scanning area; and
a display unit configured to live display the at least one tomogram,
wherein the drive unit obtains tomograms for diagnosis, from the two dimensional area, after the display unit displays the at least one tomogram.

23. The apparatus according to claim 22, wherein said driving unit drives the second optical member to obtain a tomogram from an area smaller than the two-dimensional scanning area based on central portion of the two-dimensional scanning area.
